(19) United States
(12) Patent Application Publication
(10) Pub. No.: US 2004/0116898 A1
(43) Pub. Date: Jun. 17, 2004

(54) Endotracheal tube assembly and methods of using same

(76) Inventor: William D. Hawk, Oklahoma City, OK (US)

Correspondence Address: DUNLAP, CODDING & ROGERS P.C., PO BOX 16370, OKLAHOMA CITY, OK, 73113, US

(21) Appl. No.: 10/729,080

(22) Filed: Dec. 5, 2003

Related U.S. Application Data
(60) Provisional application No. 60/431,382, filed on Dec. 5, 2002.

(51) Int. Cl. A61M 31/00; A61M 29/00; A61M 37/00; A61M 16/00; A61M 25/00

(52) U.S. Cl. 604/509; 604/103.05; 604/103.02; 128/207.15; 604/523

(57) ABSTRACT

The present invention provides an assembly and method for topically applying a local anesthetic agent into the trachea and/or hypopharynx during intubation with a tracheal tube. The present invention is designed to topically anesthetize the tracheal surfaces that are in contact with the tracheal tube, thereby preventing the patient from experiencing physiological reactions such as coughing, pain or discomfort from the indwelling tracheal tube, as well as post operative or post extubation laryngospasm. Local anesthetic application can be performed by bolus or continuous infusion.

### CROSS REFERENCE TO RELATED APPLICATIONS

This application claims benefit under 35 U.S.C. 119(e) of provisional application U.S. Serial No. 60/431,382, filed Dec. 5, 2002, the contents of which are hereby expressly incorporated herein by reference in their entirety.

### STATEMENT REGARDING FEDERALLY SPONSORED RESEARCH OR DEVELOPMENT

Not Applicable.

### BACKGROUND OF THE INVENTION

1. Field of the Invention

The present invention relates generally to apparatus and methods for topically applying local anesthetic agents into the trachea and/or hypopharynx during intubation.

2. Description of the Background Art

The necessity and advantage of endotracheal intubation in the application of anesthesia and for pulmonary therapy is well documented and widely accepted. Intubation of the trachea is necessary to protect a patient's airway during general anesthesia and to provide a way to ventilate the patient using positive pressure when the patient cannot adequately ventilate himself or herself. Intubation allows the use of positive pressure ventilation, continuous positive airway pressure and/or positive end expiratory pressure.

The common practice during-intubation is to cease ventilating the patient, insert the laryngoscope through an upper airway such as the mouth or nose, visualize the opening of the trachea, and then, under direct vision, insert a type of tracheal tube called an endotracheal tube. After this is done, ventilation is re-instituted. In certain circumstances it is necessary to form an incision in the neck region at the base of the throat and extending into the inner wall of the trachea. A tracheotomy thus allows insertion of a second type of tracheal tube, a tracheostomy tube, through the trachea wall rather than an upper airway in order to properly ventilate the lungs.

Adverse physiological and neuromuscular response to laryngoscopy and intubation remains a significant problem of airway manipulation, both during anesthesia and other airway diagnostic procedures. In airway anesthesia, manipulation of the airway either during laryngoscopy or during endotracheal intubation is often associated with laryngospasm, coughing and undesirable cardiovascular reflexes. Therefore, there are many times at the onset of an anesthetic as well as during the entire period of intubation that having the larynx, vocal cords and/or trachea topically anesthetized would avoid the sometimes dangerous side effects of laryngoscopy and intubation.

Thus, new and improved tracheal tubes and methods for topically applying local anesthetic agents into the trachea and/or hypopharynx during intubation that overcome the disadvantages and defects of the prior art are being sought.

### SUMMARY OF THE INVENTION

According to the present invention, an assembly and method for topically applying a local anesthetic agent into the trachea and/or hypopharynx of a patient during intubation with a tracheal tube are provided that overcome the disadvantages and defects of the prior art. Broadly, the present invention is designed to topically anesthetize the tracheal surfaces that are in contact with the tracheal tube, thereby preventing the patient from experiencing physiological reactions such as coughing, pain or discomfort from the indwelling tracheal tube, as well as post operative or post extubation laryngospasm. Local anesthetic application can be performed by bolus or continuous infusion.

An object of the present invention is to provide an assembly that includes a tracheal tube and a tracheal tube cover. The tracheal tube cover is sized and dimensioned so as to be positioned around the tracheal tube, and includes a sheath and at least one injection assembly. The sheath includes at least one cuff having an expandable area or space in which fluid may be disposed, and an outer surface of the cuff is constructed of a material that allows fluid disposed in the expandable space to diffuse out of the tracheal tube cover at a desired rate. The at least one injection assembly includes an injection port and tubing, wherein the tubing has a first end that is connected to the injection port and a second end that is connected to the cuff. At least a portion of the tubing of the injection assembly is disposed adjacent to the tracheal tube such that the injection port is in close proximity to the machine end portion of the tracheal tube when the tracheal tube cover is secured about the tracheal tube.

Another object of the present invention, while achieving the before-stated object, is to provide methods for topically applying a drug such as an anesthetic into the trachea and/or hypopharynx of a patient via the tracheal tube/tracheal tube cover assembly of the present invention when the patient is intubated with such assembly. The method includes providing the assembly described herein above and disposing an effective amount of an anesthetic into the at least one cuff of the tracheal tube cover through the injection assembly thereof either before or after inserting the assembly into a trachea of a patient. The anesthetic is then allowed to diffuse through the outer surface of the cuff of the tracheal tube cover over time and thereby topically anesthetize tracheal surfaces of the patient that are in contact with or in close proximity to the assembly.

Other objects, features and advantages of the present invention will become apparent from the following detailed description when read in conjunction with the accompanying drawings and appended claims.

### BRIEF DESCRIPTION OF THE DRAWINGS

FIG. 1 is a perspective view of a tracheal tube of the prior art.

FIG. 2 is a perspective view of an assembly that includes a tracheal tube and a tracheal tube cover constructed in accordance with the present invention.

FIG. 3 is a perspective view of the assembly of FIG. 2 having an anesthetic or other fluid disposed in at least one cuff of the tracheal tube cover of the present invention.

### DETAILED DESCRIPTION OF THE INVENTION

Before explaining at least one embodiment of the invention in detail by way of exemplary drawings and procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

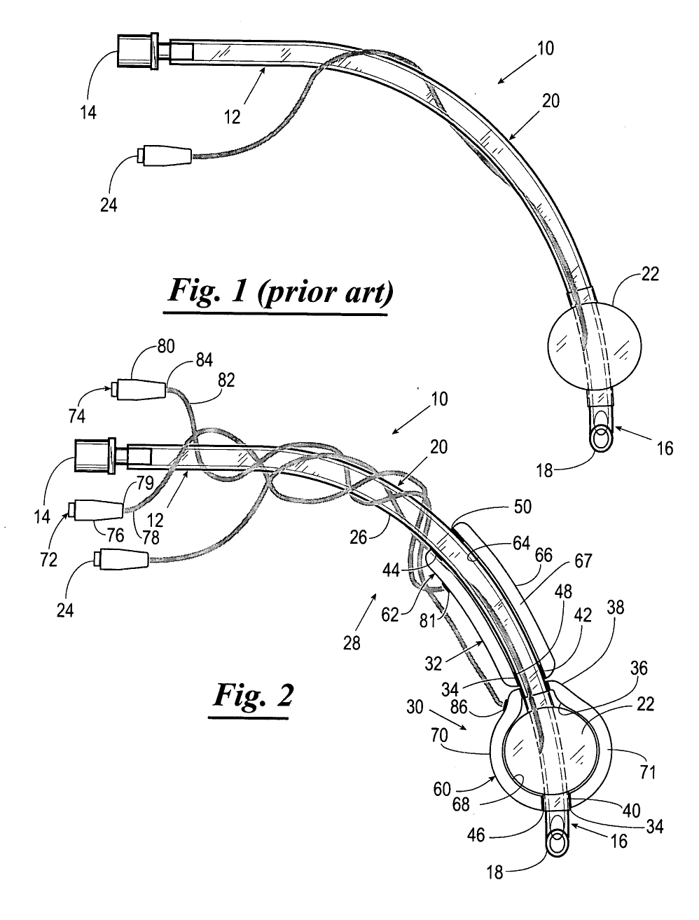

Tracheal tubes, such as but not limited to, endotracheal and tracheostomy tubes, are used by physicians for intubation into a patient's trachea for various purposes such as to enable the patient to breathe or to enable intermittent positive pressure ventilation of the respiratory tract. Various tracheal tube configurations are well known in the art. Shown in FIG. 1 is a typical tracheal tube 10 as is commonly used in endotracheal intubation. The term “tracheal tube” may be used interchangeably herein with the terms “endotracheal tube” or “tracheostomy tube”. The tracheal tube 10 typically includes a proximal or machine end portion 12 that has a terminal end 14 that is open and adapted for connection with a source of gas to provide an entry for such gas that is to be introduced into the lungs of a patient. The tracheal tube 10 also includes a distal or patient end portion 16 having a terminal end 18 that is open and typically beveled, so that the terminal end 18 provides an effluent opening for discharge of air to the trachea and lungs. An intermediate portion 20 of the tracheal tube 10 extends between and merges into the proximal or machine end portion 12 and the distal or patient end portion 16. The tracheal tube 10 is typically constructed of a shape-sustaining material that is flexible and bendable, and the intermediate portion 20 of the tracheal tube 10 is typically provided with a slight bend or curve formed therein to positionally conform approximately to the shape of a patient's posterior pharynx and trachea and extend therealong, thereby easing insertion of the tracheal tube 10 into a patient. The entire tracheal tube 10 is typically of substantially uniform internal diameter and external diameter. The internal diameter of the tracheal tube 10 is typically in a range of from about 1.0 mm to about 10 mm.

The distal or patient end portion 16 of the tracheal tube 10 is typically provided with at least one cuff 22 formed thereon (only one cuff 22 illustrated in FIG. 1 for the purposes of illustration). The cuff 22 functions as a sealing member which inflates or is inflated in order to form a seal between the tracheal tube 10 and the wall of the trachea, thereby preventing the escape of air in a direction through the trachea away from the lungs and thereby forcing and channeling the flow of air, oxygen or similar desirable gas through the trachea to inflate the lungs as is desirable. Thus, during ventilation of the patient, only air flow through a passageway formed by the internal diameter of the tracheal tube 10 occurs, thereby negating the chance for inadvertent and undesirable escape of air from the lungs. The tracheal tube 10 is also usually provided with an air injection port 24 for inflating the cuff 22. The cuff 22 is typically manufactured of a thin flexible film material such as polypropylene, polyethylene, PVC or like substantially fluid impermeable plastic film materials.

However, it is to be understood that tracheal tubes are well known and utilized in the art that are not provided with a cuff attached thereto, and thus the methods of the present invention are not limited to use with a tracheal tube having a cuff provided thereon.

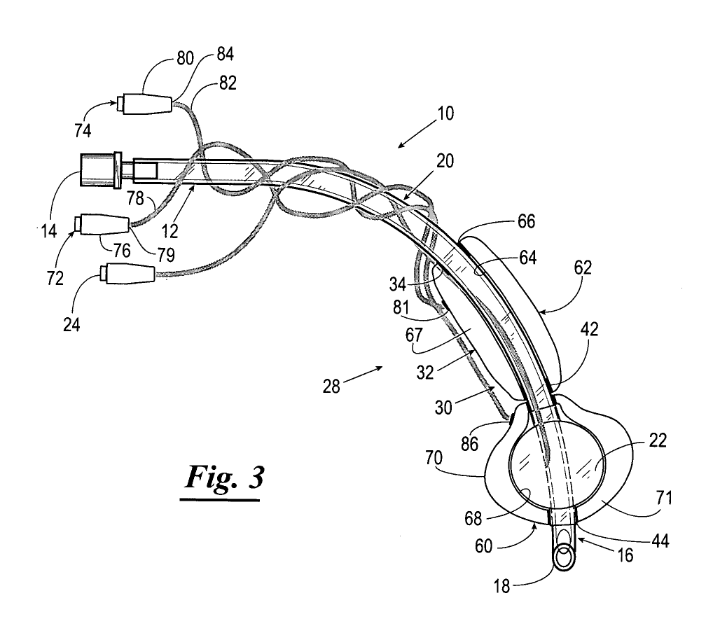

Shown in FIGS. 2 and 3 is a tracheal tube assembly 28 of the present invention which includes the tracheal tube 10 and a tracheal tube cover 30. The terms “tracheal tube cover”, “endotracheal tube cover”, “tracheal tube condom” and “endotracheal tube condom” are used herein interchangeably. The tracheal tube cover 30 may be formed separately from the tracheal tube 10 and disposed thereabout at a desired time. In another embodiment, the tracheal tube cover 30 may be integrally formed with and permanently attached to the tracheal tube 10, thus providing an assembly comprising a tracheal tube that has been modified to incorporate the elements of the tracheal tube cover 30. The term “assembly” as used herein will be understood to include a tracheal tube assembly where the tracheal tube and tracheal tube cover are separately formed, as well as a tracheal tube assembly where the tracheal tube and tracheal tube cover are integrally formed and permanently attached.

The tracheal tube cover 30 of the present invention includes at least one sheath 32 that is sized and dimensioned so as to be positioned around a typical tracheal tube, such as the tracheal tube 10 as shown, by sliding over or wrapping around the tracheal tube. For example, the sheath 32 of the tracheal tube cover 30 may be slid over the tracheal tube 10 like a condom. The tracheal tube cover 30 may be disposed about the tracheal tube 10 prior to intubation into a patient. Optionally, the tracheal tube cover 30 may be slid over the tracheal tube 10 while a patient is intubated with the tracheal tube 10.

The sheath 32 of the tracheal tube cover 30 may be held in place about the tracheal tube 10 or connected thereto by any methods known to those of ordinary skill in the art. One such method of securing the tracheal tube cover 30 about the tracheal tube 10 is shown in FIG. 2 and includes the use of a bonding material 34, such as tape. The bonding material 34 may be placed on an inner surface 36 or an outer surface 38 of the sheath 32 of the tracheal tube cover 30 in at least one location, and preferably two or more locations, and more preferably three locations. In FIG. 2, the bonding material 34 is illustrated for the purpose of example only as being placed on the inner surface 36 of the sheath 32 of the tracheal tube cover 30 at locations 40, 42 and 44 of the sheath 32, and the bonding material 34 at locations 40, 42 and 44 are disposed adjacent to and in contact with locations 46, 48 and 50 on the tracheal tube 10 for bondingly connecting the tracheal tube cover 30 to the tracheal tube 10.

When the tracheal tube cover 30 is formed separately from the tracheal tube 10, the tracheal tube cover 30 may have one or more portions that are in contact with the tracheal tube 10. That is, while the tracheal tube cover 30 may be disposed about the tracheal tube 10, the tracheal tube cover 30 may only physically contact the tracheal tube 10 at the positions where the tracheal tube cover 30 is bondingly connected or otherwise attached to the tracheal tube 10, such as for example but not by way of limitation, the locations 46 48 and 50 of the tracheal tube 10 that are in contact with locations 40, 42 and 44 of the tracheal tube cover 30. Optionally, the tracheal tube cover 30 may be disposed about the tracheal tube 10 in such a manner that the tracheal tube cover 30 is pulled taut and assumes a configuration that is closely adjacent to the configuration of the tracheal tube 10.

In addition, the tracheal tube cover 30 must be attached and secured to the tracheal tube 10 in such a manner that the tracheal tube cover 30 is at least partially sealed to the tracheal tube 10 such that when the anesthetic is disposed in the tracheal tube cover 30, the anesthetic cannot leak out from the locations at which the tracheal tube cover 30 and the tracheal tube 10 are bondingly connected.

The sheath 32 of the tracheal tube cover 30 has one or more portions or cuffs that are contained between the locations at which the tracheal tube cover 30 is bondingly connected to the tracheal tube 10. The cuff(s) provides an area or space that can expand and in which a fluid may be disposed. The tracheal tube cover 30 is illustrated in FIG. 2 as having two cuffs: a lower cuff 60 which is adapted to surround a lower portion of the tracheal tube and in particular a cuff of a tracheal tube (if provided), such as the cuff 22 of the tracheal tube 10, and an upper cuff 62 which is adapted to surround at least a portion of an intermediate portion of a tracheal tube, such as the intermediate portion 20 of the tracheal tube 10. Thus, the upper cuff 62 of the tracheal tube cover 30 is positioned on the tracheal tube 10 above the cuff 22 thereof but below the air injection port or open terminal end 14 of the proximal or machine end portion 12 of the tracheal tube 10.

The upper cuff 62 of the tracheal tube cover 30 has an inner surface 64, an outer surface 66 and an expandable area or space 67. The lower cuff 60 of the tracheal tube cover 30 has an inner surface 68, an outer surface 70 and an expandable area or space 71. The inner surfaces 64 and 68 of the upper and lower cuffs 62 and 60, respectively, may be formed from an outer surface 26 of the tracheal tube 10, or the inner surfaces 64 and 68 of the upper and lower cuffs 62 and 60, respectively, may be formed of a separate material that is adjacent to or in close proximity to the outer surface 26 of the tracheal tube 10. The outer surfaces 66 and 70 of the upper and lower cuffs 62 and 60, respectively, of the tracheal tube cover 30 are perforated, fenestrated, permeable or semi-permeable to desired fluid(s) such as but not limited to liquid or gaseous anesthetics or other drugs utilized therewith to allow the fluid to diffuse out of the upper cuff 62 and/or lower cuff 60 at a desired rate so that such fluid can come into contact with the mucous membranes of the trachea and thereby topically anesthetize the membranes. The lower cuff 60 of the tracheal tube cover 30 may be connected to and in fluid communication with the upper cuff 62, and one injection assembly may be utilized to infuse both cuffs 60 and 62. Alternatively, lower cuff 60 and the upper cuff 62 of the tracheal tube cover 30 may not be in fluid communication and thus are infused by two separate injection assemblies to allow local anesthetics to be infused separately at each location, as is illustrated in FIG. 2.

The tracheal tube cover 30 further comprises at least one injection assembly for disposing fluid, such as a liquid or gaseous anesthetic, in the cuffs 60 and 62 of the tracheal tube cover 30. In FIG. 2, the upper cuff 62 of the tracheal tube cover 30 is illustrated as being connected to an injection assembly 72, while the lower cuff 60 of the tracheal tube cover 30 is illustrated as being connected to an injection assembly 74. The injection assembly 72 includes an injection port or site 76 and tubing 78. The tubing 78 has a first end 79 that is connected to the injection port 76 and a second end 81 that is connected to the upper cuff 62. The tubing 78 is disposed adjacent to and is in close contact with the tracheal tube 10 such that the injection port 76 connected to the first end 79 of the tubing 78 lies in close proximity to the proximal or machine end portion 12 of the tracheal tube 10. The second end 81 of the tubing 78 may be attached to the upper cuff 62 at any position along the upper cuff 62.

The injection assembly 74 includes an injection port or site 80 and tubing 82. The tubing 82 has a first end 84 that is connected to the injection port 80 and a second end 86 that is connected to the lower cuff 60. The tubing 82 is disposed adjacent to and is in close contact with the tracheal tube 10 such that the injection port 80 connected to the first end 84 of the tubing 82 lies in close proximity to the proximal or machine end portion 12 of the tracheal tube 10. The second end 86 of the tubing 82 may be attached to the lower cuff 60 at any position along the lower cuff 60.

While the injection assemblies 72 and 74 are illustrated in FIG. 2 as utilizing two separate and unconnected tubings 78 and 82, it is to be understood that the two tubings 78 and 82 of injection assemblies 72 and 74 may be bonded together with two separate injection ports 76 and 80 at the end of the bonded tubings 78 and 82. In addition, the tubings 78 and 82 and/or the injection ports 76 and 80 may be labeled in some fashion to distinguish the injection assembly 72 feeding the upper cuff 62 from the injection assembly 74 feeding the lower cuff 60. For example, one tubing and/or injection port may be colored blue while the other tubing and/or injection port is colored white. One or both of the injection assemblies 72 and 74 may also be labeled to distinguish them from the air injection port 24 of the tracheal tube 10.

The sheath 32 of the tracheal tube cover 30 may be formed of any material that will allow for expansion of the one or more cuffs of the tracheal tube cover 30, such as the cuffs 60 and 62 of FIG. 2, upon insertion or injection of a fluid therein. The material from which the sheath 32 of the tracheal tube cover 30 is constructed must also be permeable or semi-permeable to the desired fluid, such as an anesthetic, to be utilized in accordance with the present invention, or the material must be capable of being perforated or fenestrated to allow the fluid to diffuse out of the material at a desired rate. Examples of polymeric materials that may be utilized in the construction of the sheath 32 of the tracheal tube cover 30 of the present invention include but are not limited to vinyl, PVC, urethane, polypropylene, polyethylene, cellophane, polystyrene, chlorinated polyethylene, cellulose nitrate, ethyl cellulose, cellulose acetate, polyvinyl butyryl, acrylic resins, alkylacrylate resins, rubber, acrylonitrile rubber, chlorinated rubber such as neoprene, and combinations or laminations thereof.

The fluid utilized in accordance with the present invention may be a liquid or a gas. In a preferred embodiment, the assembly of the present invention is utilized with an anesthetic. Anesthetics that may be utilized in accordance with the tracheal tube cover 30 include, but are not limited to, lidocaine, dibucaine, prilocaine, novocaine, other anesthetics of this class or combinations or mixtures thereof. In addition, while the tracheal tube cover 30 has been described herein for use with anesthetics, it is to be understood that any drug for which administration is desired in this manner may be utilized with the assembly and methods of the present invention, and therefore fall within the scope of the present invention.

In one method of use of the present invention, as shown in FIGS. 2 and 3, the tracheal tube assembly 28 is provided, or the tracheal tube cover 30 is disposed about a tracheal tube, such as the tracheal tube 10, by any of the methods described herein or known to those of ordinary skill in the art. For example, the tracheal tube cover 30 may be slid around the tracheal tube 10, or the tracheal tube cover 30 may be wrapped around the tracheal tube 10. When the method involves disposing the tracheal tube cover 30 about the tracheal tube 10, the ends of the tracheal tube cover 30 disposed about the tracheal tube 10 are then secured to the tracheal tube 10 to seal the tracheal tube cover 30 about the tracheal tube 10 by any methods described herein or known to those of ordinary skill in the art, such as by bondingly connecting a portion of the tracheal tube cover 30 to the tracheal tube 10. The tracheal tube cover 30 is secured and sealed about the tracheal tube 10 such that any fluid disposed in the cuffs of the tracheal tube cover 30 will not leak out of the end positions at which the tracheal tube cover 30 and the tracheal tube 10 are bondingly connected.

An effective amount of an anesthetic is then disposed in the cuff(s) of the tracheal tube cover, such as the cuffs 60 and 62 of the tracheal tube cover 30, via the injection assemblies 72 and 74. The term “effective amount of an anesthetic” will be understood herein to include an amount of anesthetic sufficient to diffuse through the outer surface of the cuff of the tracheal tube cover and anesthetize the tracheal surfaces that are in contact with the tracheal tube. Such “effective amount of an anesthetic” may be an amount of anesthetic sufficient to provide a bolus of anesthetic or a continuous infusion of anesthetic.

As illustrated in FIG. 3, an anesthetic or other fluid is disposed through the injection assembly 72 of the upper cuff 62 of the tracheal tube cover 30. This causes the anesthetic or other fluid to be disposed between the inner and outer surfaces 62 and 64, respectively, of the upper cuff 62, and therefore at least partially fill and thus expand the expandable space 67 of the the upper cuff 62. Likewise, an anesthetic or other fluid may be disposed through the injection assembly 74 of the lower cuff 60 of the tracheal tube cover 30, causing the anesthetic or other fluid to be disposed between the inner and outer surfaces 68 and 70, respectively, of the lower cuff 60, and therefore at least partially filling and expanding the expandable space 71 of the lower cuff 60.

The assembly 28 shown in FIG. 3 and having the anesthetic or other fluid disposed in at least one of the cuffs of the tracheal tube cover 30 may now be inserted into a patient by methods well known in the art. In an alternative embodiment, the assembly 28 shown in FIG. 2 may be inserted into a patient by methods well known in the art, and the anesthetic or other fluid is then disposed in the tracheal tube cover 30 as described herein above following insertion of the assembly 28 into the patient.

In yet another embodiment of the present invention, the tracheal tube 10 may first be inserted into a patient by methods well known in the art, and once the tracheal tube 10 is in place, the tracheal tube cover 30 is then slid over the tracheal tube 10 to provide the tracheal tube assembly 28 in the intubated patient. For example, a stylet may be utilized that allows sliding the tracheal tube cover 30 over the intubated tracheal tube 10 like a condom, and then the stylet is removed. Once the tracheal tube assembly 28 is in place, the anesthetic or other fluid may be disposed in the tracheal tube cover 30 as described herein above.

Over a desired amount of time, the anesthetic or other fluid diffuses through the outer surface(s) of the cuff(s) of the tracheal tube cover, such as the outer surfaces 66 and 70 of the upper and lower cuffs 62 and 60, respectively, of the tracheal tube cover 30, and thus topically anesthetizes the tracheal surfaces that are in contact with or in close proximity to the tracheal tube. The assembly and method of the present invention described herein will prevent the patient from experiencing physiological reactions such as coughing, pain or discomfort from the indwelling tracheal tube, as well as post operative or post extubation laryngospasm.

In addition, the anesthetic application can be performed by bolus or continuous infusion. That is, a bolus of anesthetic may be infused to one or more of the cuffs 60 and 62 of the tracheal tube cover 30 at desired intervals via the injection assemblies 72 and 74, or the injection assemblies 72 and 74 may be utilized to refill the supply of anesthetic or other fluid provided in the retaining space of each cuff.

Thus, in accordance with the present invention, there has been provided an assembly and method for topically applying a local anesthetic agent into the trachea and/or hypopharynx during intubation with a tracheal tube that fully satisfies the objectives and advantages set forth herein above. Although the invention has been described in conjunction with the specific drawings and language set forth herein above, it is evident that many alternatives, modifications, and variations will be apparent to those skilled in the art. Accordingly, it is intended to embrace all such alternatives, modifications and variations that fall within the spirit and broad scope of the invention.

What is claimed is:

1. A tracheal tube cover sized and dimensioned so as to be positioned around a tracheal tube wherein the tracheal tube comprises a machine end portion and a patient end portion, the tracheal tube cover comprising:
a sheath comprising at least one cuff having an expandable space in which fluid may be disposed, an outer surface of the at least one cuff being constructed of a material that allows fluid disposed in the expandable space to diffuse out of the tracheal tube cover at a desired rate; and
at least one injection assembly comprising an injection port and tubing, the tubing having a first end that is connected to the injection port and a second end that is connected to the at least one cuff, wherein at least a portion of the tubing is disposed adjacent to the tracheal tube such that the injection port is in close proximity to the machine end portion of the tracheal tube when the tracheal tube cover is secured about the tracheal tube.

2. The tracheal tube cover of claim 1 further comprising a bonding material for connecting the tracheal tube cover to the tracheal tube whereby the tracheal tube cover is secured about the tracheal tube.

3. The tracheal tube cover of claim 1 wherein the tracheal tube cover is provided with an upper cuff and a lower cuff.

4. The tracheal tube cover of claim 3 wherein the upper cuff is adapted to surround at least a portion of an intermediate portion of the tracheal tube below an air injection port of the machine end portion of the tracheal tube.

5. The tracheal tube cover of claim 3 wherein the lower cuff is adapted to surround a cuff of the tracheal tube.

6. The tracheal tube cover of claim 3 wherein the upper cuff is in fluid communication with the lower cuff, and one injection assembly is used to infuse both cuffs.

7. The tracheal tube cover of claim 3 wherein the upper cuff is connected to a first injection assembly and the lower cuff is connected to a second injection assembly.

8. The tracheal tube cover of claim 7 wherein the tubing of at least one of the first and second injection assemblies is labeled or colored to distinguish between the first injection assembly and the second injection assembly.

9. A method for topically anesthetizing tracheal surfaces of an intubated patient, comprising the steps of:
providing an assembly comprising:
a tracheal tube having a machine end portion and a patient end portion;
a tracheal tube cover disposed and sealed about the tracheal tube, the tracheal tube cover comprising:
a sheath comprising at least one cuff having an expandable space in which fluid may be disposed, an outer surface of the at least one cuff being constructed of a material that allows fluid disposed in the expandable space to diffuse out of the tracheal tube cover at a desired rate; and
at least one injection assembly comprising an injection port and tubing, the tubing having a first end that is connected to the injection port and a second end that is connected to the at least one cuff, wherein at least a portion of the tubing is disposed adjacent to the tracheal tube such that the injection port is in close proximity to the machine end portion of the tracheal tube when the tracheal tube cover is secured about the tracheal tube;
disposing an effective amount of an anesthetic in the at least one cuff of the tracheal tube cover through the injection assembly of the tracheal tube cover;
inserting the assembly into a trachea of a patient; and
allowing the anesthetic to diffuse through the outer surface of the at least one cuff of the tracheal tube cover over time and thereby topically anesthetize the tracheal surfaces of the patient that are in contact with or in close proximity to the assembly.

10. The method of claim 9 wherein the step of providing an assembly further comprises connecting the tracheal tube cover to the tracheal tube via a bonding material whereby the tracheal tube cover is secured about the tracheal tube.

11. The method of claim 9 wherein, in the step of providing the assembly, the tracheal tube cover is provided with an upper cuff and a lower cuff.

12. The method of claim 11 wherein the upper cuff is adapted to surround at least a portion of an intermediate portion of the tracheal tube below an air injection port of the machine end portion of the tracheal tube.

13. The method of claim 11 wherein the lower cuff is adapted to surround a cuff of the tracheal tube.

14. The method of claim 11 wherein the upper cuff is in fluid communication with the lower cuff, and one injection assembly is used to infuse both cuffs.

15. The method of claim 11 wherein the upper cuff is connected to a first injection assembly and the lower cuff is connected to a second injection assembly.

16. The method of claim 15 wherein the tubing of at least one of the first and second injection assemblies is labeled or colored to distinguish between the first injection assembly and the second injection assembly feeding the second cuff.

17. A method for topically anesthetizing tracheal surfaces of an intubated patient, comprising the steps of:
providing an assembly comprising:
a tracheal tube having a machine end portion and a patient end portion;
a tracheal tube cover disposed and sealed about the tracheal tube, the tracheal tube cover comprising:
a sheath comprising at least one cuff having an expandable space in which fluid may be disposed, an outer surface of the at least one cuff being constructed of a material that allows fluid disposed in the expandable space to diffuse out of the tracheal tube cover at a desired rate; and
at least one injection assembly comprising an injection port and tubing, the tubing having a first end that is connected to the injection port and a second end that is connected to the at least one cuff, wherein at least a portion of the tubing is disposed adjacent to the tracheal tube such that the injection port is in close proximity to the machine end portion of the tracheal tube when the tracheal tube cover is secured about the tracheal tube;
inserting the assembly into a trachea of a patient;
disposing an effective amount of an anesthetic in the at least one cuff of the tracheal tube cover through the injection assembly of the tracheal tube cover; and
allowing the anesthetic to diffuse through the outer surface of the at least one cuff of the tracheal tube cover over time and thereby topically anesthetize the tracheal surfaces of the patient that are in contact with or in close proximity to the assembly.

18. The method of claim 17 wherein the step of providing an assembly further comprises connecting the tracheal tube cover to the tracheal tube via a bonding material whereby the tracheal tube cover is secured about the tracheal tube.

19. The method of claim 17 wherein, in the step of providing the assembly, the tracheal tube cover is provided with an upper cuff and a lower cuff.

20. The method of claim 19 wherein the upper cuff is adapted to surround at least a portion of an intermediate portion of the tracheal tube below an air injection port of the machine end portion of the tracheal tube.

21. The method of claim 19 wherein the lower cuff is adapted to surround a cuff of the tracheal tube.

22. The method of claim 19 wherein the upper cuff is in fluid communication with the lower cuff, and one injection assembly is used to infuse both cuffs.

23. The method of claim 19 wherein the upper cuff is connected to a first injection assembly and the lower cuff is connected to a second injection assembly.

24. The method of claim 23 wherein the tubing of at least one of the first and second injection assemblies is labeled or colored to distinguish between the first injection assembly and the second injection assembly feeding the second cuff.

25. A tracheal tube assembly comprising:
a tracheal tube having a machine end portion and a patient end portion; and
a tracheal tube cover disposed and sealed about the tracheal tube, the tracheal tube cover comprising:
a sheath comprising at least one cuff having an expandable space in which fluid may be disposed, an outer surface of the at least one cuff being constructed of a material that allows fluid disposed in the expandable space to diffuse out of the tracheal tube cover at a desired rate; and
at least one injection assembly comprising an injection port and tubing, the tubing having a first end that is connected to the injection port and a second end that is connected to the at least one cuff, wherein at least a portion of the tubing is disposed adjacent to the tracheal tube such that the injection port is in close proximity to the machine end portion of the tracheal tube when the tracheal tube cover is secured about the tracheal tube.

26. The tracheal tube assembly of claim 25 wherein the tracheal tube and the tracheal tube cover are integrally formed and permanently attached.

27. The tracheal tube assembly of claim 25 wherein the tracheal tube and tracheal tube cover are formed separately.

28. The tracheal tube assembly of claim 25 wherein the tracheal tube cover is provided with an upper cuff and a lower cuff.

29. The tracheal tube assembly of claim 28 wherein the upper cuff is adapted to surround at least a portion of an intermediate portion of the tracheal tube below an air injection port of the machine end portion of the tracheal tube.

30. The tracheal tube assembly of claim 28 wherein the lower cuff is adapted to surround a cuff of the tracheal tube.

31. The tracheal tube assembly of claim 28 wherein the upper cuff is in fluid communication with the lower cuff, and one injection assembly is used to infuse both cuffs.

32. The tracheal tube assembly of claim 28 wherein the upper cuff is connected to a first injection assembly and the lower cuff is connected to a second injection assembly.

33. The tracheal tube assembly of claim 32 wherein the tubing of at least one of the first and second injection assemblies is labeled or colored to distinguish between the first injection assembly and the second injection assembly.

34. A method for topically anesthetizing tracheal surfaces of an intubated patient, comprising the steps of:
providing a tracheal tube having a machine end portion and a patient end portion;
inserting the tracheal tube into a trachea of a patient;
providing a tracheal tube cover comprising:
a sheath comprising at least one cuff having an expandable space in which fluid may be disposed, an outer surface of the at least one cuff being constructed of a material that allows fluid disposed in the expandable space to diffuse out of the tracheal tube cover at a desired rate; and
at least one injection assembly comprising an injection port and tubing, the tubing having a first end that is connected to the injection port and a second end that is connected to the at least one cuff, wherein at least a portion of the tubing is disposed adjacent to the tracheal tube such that the injection port is in close proximity to the machine end portion of the tracheal tube when the tracheal tube cover is secured about the tracheal tube;
disposing the tracheal tube cover about the tracheal tube inserted into the trachea of the patient;
disposing an effective amount of an anesthetic in the at least one cuff of the tracheal tube cover through the injection assembly of the tracheal tube cover; and
allowing the anesthetic to diffuse through the outer surface of the at least one cuff of the tracheal tube cover over time and thereby topically anesthetize the tracheal surfaces of the patient that are in contact with or in close proximity to the assembly.

35. The method of claim 34 wherein, in the step of providing the assembly, the tracheal tube cover is provided with an upper cuff and a lower cuff.

36. The method of claim 35 wherein the upper cuff is adapted to surround at least a portion of an intermediate portion of the tracheal tube below an air injection port of the machine end portion of the tracheal tube.

37. The method of claim 35 wherein the lower cuff is adapted to surround a cuff of the tracheal tube.

38. The method of claim 35 wherein the upper cuff is in fluid communication with the lower cuff, and one injection assembly is used to infuse both cuffs.

39. The method of claim 35 wherein the upper cuff is connected to a first injection assembly and the lower cuff is connected to a second injection assembly.

40. The method of claim 39 wherein the tubing of at least one of the first and second injection assemblies is labeled or colored to distinguish between the first injection assembly and the second injection assembly feeding the second cuff.
